(19) United States
(12) Patent Application Publication
(10) Pub. No.: US 2024/0228416 A1
(43) Pub. Date: Jul. 11, 2024

(54) TERPINEOL WITH CONTROLLED OPTICAL ROTATION FROM GUM TERPENTINE OIL AND PROCESS THEREOF

(71) Applicant: HIMACHAL TEREPENE PRODUCTS PVT. LTD., HIMACHAL PRADESH (IN)

(72) Inventor: Akhil MAHESHWARI, HIMACHAL PRADESH (IN)

(73) Assignee: HIMACHAL TEREPENE PRODUCTS PVT. LTD., HIMACHAL PRADESH (IN)

(21) Appl. No.: 18/558,227

(22) PCT Filed: Apr. 29, 2022
(86) PCT No.: PCT/IN2022/050407

§ 371 Date: Oct. 31, 2023

(30) Foreign Application Priority Data
May 1, 2021 (IN) 202111020017

(51) Int. Cl. C07C 33/14

(52) U.S. Cl. CPC C07C 33/14; C07C 2601/16

(57) ABSTRACT

The present invention discloses an industrial process for the manufacturing of Terpineol with controlled optical rotation range i.e., −12° to −14°o. The Terpineol production process comprises of various steps including, separation of alpha pinene from gum turpentine oil, reaction of alpha pinene with Orthophosphoric acid under a temperature ranging from 50 to 60° C. for about 18-20 hours in order to produce crude terpineol, Further, the crude terpineol is subjected to distillation for about 50 to 60 hours to separate final product terpineol. Finally, the boiling point and optical rotation of the extracted product is measured. The product is obtained with a yield>90% and with purity greater than 85%.

### FIELD OF INVENTION

The invention generally relates to Terpineol obtained from Gum turpentine oil. More specifically, it pertains to Terpineol with controlled Optical rotation value/Laevo-symmetry isomeric (optical) activity from Gum turpentine oil and the process thereof.

### BACKGROUND OF THE INVENTION

### Terms and Definitions

Optical Rotation: Optical rotation is defined as rotation of the orientation of the plane of polarization about the optical axis of linearly polarized light as it travels throughout certain material. (https://en.wikipedia.org/wiki/Optical_rotation)

Dextrorotatory: When optically active substances rotate plane polarized light to the right. (https://www.sciencedirect.com/topics/biochemistry-genetics-and-molecular-biology/optical-rotation)

Laevorotatory: When optically active substances rotate plane polarized light to the left. (https://www.sciencedirect.com/topics/biochemistry-genetics-and-molecular-biology/optical-rotation)

Polarimeter: It is an instrument, used to measure optical rotation. It produces plane polarized light and make it pass throughout the liquid/sample and further measures the angle through which the plane of vibration of the plane polarized light is rotated. It detects whether one or both enantiomers are present in a sample. (https://www.sciencedirect.com/topics/biochemistry-genetics-and-molecular-biology/optical-rotation)

Turpentine:

Turpentine which is also known as Spirit of turpentine, oil of turpentine, terebenthene etc. is basically a fluid obtained by the distillation of resin harvested from living trees mainly pines (https://en.wikipedia.org/wiki/Turpentine). Turpentine is basically an essential oil obtained from pine trees and is a mixture of various monoterpenes hydrocarbons.

Turpentine is widely used as a solvent in chemical industries, resin and in paints, varnishes and coatings. According to National toxicology program, NIH, Turpentine produced in U.S. comprises of 75-85% of α-pinene with varying amount of β-pinene, camphene, 3-carene, limonene and terpinolene.

(https://ntp.niehs.nih.gov/ntp/htdocs/chem_background/exsumpdf/turpentine_508)

Alpha Pinene and Terpineol:

Alpha pinene is an organic compound of class Terpene and can be obtained by vacuum filtration distillation of turpentine. The hydration of alpha pinene in presence of acid catalyst results in the formation of Terpineol. Terpineol is a monocyclic monoterpenoid alcohol. Terpineol exists in form of four structural isomers, namely, α-terpineol, β-terpineol, γ-terpineol and 4-terpineol, out of which α-terpineol predominates. A-terpineol is a racemic mixture of (R)-(+)- and (S)-(−)-enantiomers. (https://www.acs.org/content/acs/en/molecule-of-the-week/archive/t/alpha-terpineol.html)

Alpha Terpineol is most widely used in fragrant substances in cosmetic industry, as a disinfectant and odorant in cleaning industry, Mineral floatation agent, apart from these, Terpineol also possess certain biological properties such as Antioxidant, Anti-inflammatory, anticonvulsant, antifungal, antimicrobial and ant carcinogenic properties. (https://www.researchgate.net/publication/274746922)

The basic methodology for Terpineol extraction involves the hydration of alpha pinene or turpentine crude oil in present of aqueous mineral acid catalyst.

Another process is Biotechnological process which involves the biotransformation of alpha pinene into alpha Terpineol with the help of certain microorganisms such as Pseudomonas, Aspergillus niger, Absidia corulea etc. (https://www.researchgate.net/publication/341739951.

### Significance of Optical Rotation Range of Present Invention

Optical rotation is the semi qualitative test for purity of the compound of interest, which helps to evaluate the quality of drug compound. This may be explored both form levo symmetry and dextro symmetry optical activity. Lower optical activity indicates that less impurity is present in the compound.

Most of the prior art process used for production of terpineol usually result in production of terpineol with varying optical rotation value such as −40 to −71 at higher side and −6 to −7 at lower side. Even some of the processes also result in production of terpineol with positive optical rotation value which is under dextro symmetry optical activity.

Usually at these ranges, the product does not remain constant for a longer period of time and as a result, the shelf life and stability of the product also decreases. Moreover, the prior art processes for terpineol production involves the usage of a large amount of water for the hydration of alpha pine from turpentine oil.

### INNOVATIVE APPROACH USED BY THE INVENTORS

Present invention overcomes these drawbacks and offers technical and economic advantages not offered by any of the prior art processes. The present invention discloses simple and eco-friendly industrial process for manufacturing of Terpineol with controlled Optical rotation value/Laevo-symmetry isomeric (optical) activity from Gum turpentine oil. The optical rotation of the compound produced in present invention is very low −12° to −14° as compare to products manufactured by other vendors who have worked on optical rotation capability of compounds.

### Prior Art

Patent Application No: U.S. Pat. No. 2,898,380A Discloses “Production of alpha Terpineol”.

The above invention discloses a method for the separation of alpha pinene from alpha pinene containing material which includes reacting alpha pinene containing material to sulphuric acid, to form a crude agglomerated reaction mixture containing terpine hydrate, occluded sulfuric acid, unreacted pinene-containing materials and oily by-products, removing a substantial portion of the sulphuric acid, and then dehydrating the terpin hydrate to form alpha-terpineol, the improvement which comprises treating the crude agglomerated reaction mixture with a small amount of a hydrocarbon solvent, sufficient to reduce the viscosity of the crude reaction mixture and to break up the agglomerates therein, and then washing with aqueous media to effectively reduce the occluded sulphuric acid content to a predetermined maximum concentration for the subsequent dehydration of the terpin hydrate to alpha-Terpineol, whereby the necessity of isolating and purifying the terpin hydrate is obviated.

The present invention differs from the above invention, as the present invention involves the separation of Terpineol with a specific optical rotation range −12 to −14 from alpha pinene using distillation process and by using Orthophosphoric acid, whereas the present invention discloses a method of separation of alpha pinene using sulphuric acid.

Patent No: CN102225885B Discloses “Method for increasing optical purity of alpha-Terpineol”

The above invention discloses a method for increasing optical purity of alpha Terpineol. The process uses a racemate of alpha-Terpineol or alpha-Terpineol with low optical activity as a raw material, and cheap chiral dehydroabietylamine as a resolving agent, and performs resolution by an inclusion resolution method to obtain both levorotary alpha-Terpineol and a certain amount of dextral alpha-Terpineol. The specific rotatory power [α] of the left-handed alpha-Terpineol of fractionation product that obtains _{D}²⁰Value is −40° to −70°, and the e.e value is 38˜65%, and yield is 65˜72%; The specific rotatory power of dextrorotation alpha-Terpineol [α] D20 value is +45° to +75°, and the e.e value is 41˜70%, and yield is 45˜55%.

The present invention differs from the above cited invention, as in present invention, modified distillation process is used for the separation of Terpineol with optical rotation activity −12° to −14° from alpha pinene, whereas above invention utilizes Inclusion resolution method to obtain both levorotary alpha Terpineol and a certain amount of dextral alpha Terpineol with optical rotation value of _{D}²⁰Value is −40° to −70° and +45° to +75° respectively.

Patent No: CN100357240C Discloses “Process for Preparing Laevogyrate alpha-Terpineol”

The above invention discloses, a kind of preparation method of left-handed alpha-terpineol based on the turpentines that contains α-Pai Xi is raw material, and low-carbon alcohol as a solvent, and Zeo-karb is a catalyzer, produces left-handed alpha-Terpineol by fixed-bed reactor single stage method hydration reaction. The above invention involves the use of Cation exchange resin, 1.2% Liquid ferri trichloridi for the preparation of catalyst. The process of synthesis of left handed alpha Terpineol is conducted in tubular fixed bed reactor. The synthesis involves the use of turpentine, water and n-propyl alcohol, the solution is heated, heating results in evaporation of firpene, water and alcohol. Further after condenser condenses and reacts, refluxing then enters three-necked flask, continuously circulation, the fixed bed reaction actuator temperature is kept about 90° C., and reaction takes place for about 9 hours. When reaction completes, the alcohol is removed by the process of simple distillation. And this results in the formation of left handed alpha Terpineol with 65-75%, yield and with optical rotation of α²⁵_{D}=−21.1° to α²⁵_{D}=−23.6°.

The present invention involves the use of Orthophosphoric acid for separation of Terpineol from alpha pinene under a temperature ranging for 50-60° C. and the reaction get completed in approximately 18-20 hours. Further the crude Terpineol undergoes distillation for about 50 to 60 hours in order to separate pure Terpineol. Whereas above invention involves the use of a completely different methodology. Also, present invention produces a product with 85-90% purity and optical rotation ranging from −12° to −14° whereas in above invention, left handed alpha Terpineol is produced with 65-75% yield and with optical rotation of α²⁵_{D}=−21.1° to α²⁵_{D}=−23.6°. Hence present invention differs from the above cited patent.

Patent No: CN102225886B Discloses “Method of preparing alpha Terpineol with high optical purity”

The above invention discloses a method for preparation of alpha Terpineol using a resolving agent. Inclusion resolution method is used for obtaining high optical purity of alpha Terpineol. By using method disclosed in above invention, the specific rotatory power of the left-handed alpha-Terpineol of product [α]_{D}²⁰=−90°˜−99°, the enantiomeric excess value surpasses 85%, and average yield is 62˜70% and The specific rotatory power of dextrorotation alpha-Terpineol [α]_{D}²⁰=+90°˜+99°, the enantiomeric excess value surpasses 85%, and average yield is 60˜68% is obtained.

The present invention differs from the above cited invention, as in present invention, modified distillation process is used for the separation of Terpineol with optical rotation activity −12° to −14° from alpha pinene, whereas above invention utilizes Inclusion resolution method to obtain both levorotary alpha Terpineol and a certain amount of dextral alpha Terpineol with optical rotation value of _{D}²⁰Value is −90° to −99° and +90° to +99° respectively.

Patent Application No: DE4111900A1 Discloses Optically Active Alpha Terpineol Production by Hydration of Optically active Alpha and/or beta pinene using specified acid mixture.

The Above invention relates to a method for producing optically active α-Terpineol from optically active α- and/or β-pinene or from oils containing them such as Turpentine oil while maintaining the optical configuration/optical direction of rotation. The above process involves the use of water, 10% sulphuric acid, and Acetic acid and American Turpentine oil. The raw materials are filled in 21 stirrer and heated at 100° C., reflux for 4 hours. The aqueous phase after reaction is separated and the organic phase is washed with 80 g of water and buffered with 50% sodium hydroxide. The final products are further analyzed using Gas chromatography and finally Terpineol is separated.

The present invention involves the use of Orthophosphoric acid for conversion of alpha pinene to Terpineol under temperature 50-60° C. for almost 18-20 hours. Whereas in above invention, sulphuric acid, acetic acid etc are used. Also present invention results in production of Terpineol with optical rotation ranging from −12° to −14 Hence present invention differs from the above cited invention.

Patent No: CN103497084B Discloses “Method of producing alpha pinene from Turpentine”

The above invention discloses a method for producing alpha pinene from Turpentine. The method comprises the following steps: feeding; vacuumizing; predistilling; refluxing; distilling off alpha-pinene; carrying out repeated distillation; distilling off beta-pinene; distilling off bipentene; and collecting heavy oil. The preparation method has the advantages of higher production stability and higher reaction safety, and can effectively separate the alpha-pinene, beta-pinene, bipentene and heavy oil from turpentine.

The present invention, alpha pinene is first separated from turpentine and is then reacted with Orthophosphoric acid to extract Terpineol with optical rotation ranging from −12° to −14°

### Journals and Publications

Busi Siddhardha, M.Vijay kumar, U.S.N Murty, G.S. Ramanjaneyulu and S.prabhakar: Biotransformation of α-pinene to Terpineol by Resting cell Suspension of Absidia corulea: Indian Journal of Microbiology: 2012 Jun; 52(2): 292-294

Above publication discloses biotransformation of α-pinene to Terpineol with the help of Resting cell suspension of Absidia corulea (Fungus) and further measurement of the optical activity value of the extracted Terpineol. In above publication, after Extraction, the crude product i.e., Terpineol is analysed and purified using GC-MS and Column chromatography. Further the optical rotation value of purified Terpineol was measured with JASCO-DIP-360 Polarimeter, which was found to be [α]_{D}²⁰=−258 °

The present invention differs from the above publication, as in above publication, a biological microorganism i.e., fungus Absidia corulea is used for the biotransformation of α-pinene to Terpineol, whereas in present invention, modified distillation process is used for the separation of Terpineol from alpha pinene. Also in above publication, the optical rotation value of purified Terpineol is maintained as −258°, whereas in present invention, Terpineol is extracted with range of −12 to −14°. Hence present invention differs from the above publication.

Tirto Prakoso, Ilham Ardiyanto Putra, Lienda Handojo Tatang Hernas Soerawidjaja, Haryo Pandu Winoto, Antonius Indarto: a method to control Terpineol production from turpentine by acid catalysts mixing: Heliyon Elsevier: March, 2020

The above publication investigates the effect of single/Individual and Mixed/Combined catalysts for Terpineol yield. In the above publication, Mix of turpentine oil, demineralized water and acid catalyst are heated and stirred in a reactor. The reaction mixture results in formation of two layers of liquid and further separation was conducted using a separation funnel. The top layer comprises of residual turpentine by products and Terpineol and the bottom layer comprises of acid impurities and water residues. After removal of impurities, the final product in above publication was analysed using Gas chromatograph-Mass Spectroscopy.

The present invention involves a process for separation of Terpineol with a specific optical rotation range of −12 to −14 from alpha pinene separated from Gum turpentine oil. In the present invention, ortho phosphoric acid is used as acid catalyst, whereas in above publication, the effect of single/Individual catalyst and Mixed/Combined catalyst is determined for Terpineol yield. Also present invention deals with manufacturing of Terpineol with a specific optical rotation range, whereas the above publication does not discloses any specific optical rotation range of extracted final product.

Adones sales, Loren da oliveria felipe, Juliano Lemos Bicas: Production, Properties and Applications of alpha Terpineol; 2020; Food and Bioprocess Technology an International Journal.

The above publication discloses the properties, production methods and Application of alpha Terpineol in various fields. The above publication discloses chemical and biotechnological methods for the synthesis of alpha Terpineol from turpentine oil. According to above publication, the classical process to produce alpha Terpineol involves the hydration of alpha pinene or turpentine crude oil with aqueous mineral acid and further produced turpentine hydrate is converted to desired product by partial dehydration. Moreover, many bioprocesses for the microbial production of α-Terpineol via biotransformation of monoterpenes (limonene, α- and β-pinene) are also disclosed in the above publication. In above publication, left handed α-terpineol with −35.5° to −110.3° at 25° C. was produced.

The present invention involves a process for separation of Terpineol with a specific optical rotation range of −12 to −14 from alpha pinene separated from Gum turpentine oil, which is not disclosed in the above publication, hence present invention differs from the above cited publication.

Rui Feng Mei, Ya Xian Shi, Wei-He Duan, Hao Ding, Xiao-Ran Zhang, Le Cai and Zhong-Tao Ding: Biotransformation of α-Terpineol by Alternaria alternate: Royal Society of Chemistry: 2020, 10, 6491-6496

The Above publication discloses the method of biotransformation of alpha Terpineol. In this study, alpha Terpineol was biotransformed to 4R-oleuropeic acid and (1S, 2R, 4P)-P-menthane-1,2,8-triol by oxidation reaction. The above process uses 7-hydroxy-alpha-terpineol with optical rotation of [α]²⁵_{D} −25.1°.

The present invention involves a process for separation of Terpineol with a specific optical rotation range of −12 to −14 from alpha pinene separated from Gum turpentine oil, which is not disclosed in the above publication, hence present invention differs from the above cited publication.

A.Aguilar Elguezabal, L. De La Torre-Saenz, M.Rpman-Aguirre, L.Alvarez-contreras: Ionic Liquid as Green Solvent for the Synthesis of alpha-Terpineol from alpha pinene: Sustainable chemistry and Pharmacy: volume 15, March 2020, 100207.

The above publication discloses novel greener approach to transform α-pinene to α-terpineol by the use of a low cost ionic liquid that acts as solvent, maintaining the water and acid catalyst that pinene hydration reaction requires, in the same inorganic phase, once reaction has finished and product is separated by simple decantation. By this method of α-terpineol synthesis from α-pinene, the neutralization of product is avoided, and also the necessity to use new catalyst each reaction batch or the recovery of a solvent by distillation.

The present invention involves a process for separation of Terpineol with a specific optical rotation range of −12 to −14 from alpha pinene, which is not disclosed in above citation. Hence, the present invention differs from the above cited paper.

### Commercial Information

### Alpha-Terpineol (Compound)

https://pubchem.ncbi.nlm.nih.gov/compound/alphaTERPINEOL#section=Stability-Shelf-Life

The Above mentioned Alpha Terpineol has a melting point of 37.0° C., Boiling point of 218-221° C. and Optical Rotation value of 106.4 deg at 20° C./D.

The Terpineol manufactured by present process has optical rotation value of −12 to −14° C. hence differs from the above mentioned product.

### Alpha-Terpineol

https://www.drugfuture.com/chemdata/alpha-terpineol.html

D-form of the above mentioned alpha Terpineol has an optical rotation value of [a] D20 +92.45°.

The Terpineol manufactured by present process has optical rotation value of −12 to −14° C., hence differs from the above mentioned product.

### (S)-(-)-α-Terpineol

https://www.merckmillipore.com/IN/en/product/S-Terpineol,MDA_CHEM 821078?ReferrerURL=https%3A%2F%2Fwww.google.com%2F

The above mentioned product, CAS 10482-56-1, molar mass 154.25 g/mol, has an optical rotation value of [α] 20/D −102.5 to −97.5°.

The Terpineol manufactured by present process has optical rotation value of −12 to −14° C., hence differs from the above mentioned product.

Therefore it is clear from the above that others have made compound Terpineol with varying optical rotation value such as −40 to −71 at higher side and −6 to −7 at lower side. But the process of present invention has resulted in compound Terpineol with very low optical values of −12° to −14°

The product of specific optical rotation value OF −12° TO −14° remains constant for a longer period of time Hence effectiveness of the products prepared from extracted terpineol will increase.

### OBJECT OF THE PRESENT INVENTION

The main object of the present invention is to disclose an industrial process for manufacturing of terpineol from gum turpentine oil with controlled optical rotation value i.e., −12° to −14°.

One more object of the present invention is to disclose a simple, eco-friendly and commercially viable process for extraction of Terpineol.

### SUMMARY OF THE INVENTION

The present invention discloses an industrial process for the manufacturing of Terpineol from gum turpentine oil with controlled optical rotation value i.e., −12° to −14°. Terpineol is basically separated from alpha pinene extracted from gum turpentine oil. The process disclosed in present invention comprises of various steps including, Separation of alpha pinene from gum turpentine oil, Reaction of alpha pinene with Orthophosphoric acid under temperature ranging from 50 to 60° C. for about 18 to 20 hours, in order to produce crude terpineol, further the crude terpineol is subjected to distillation for 50 to 60 hours to separate final product Terpineol. Finally, the boiling point and optical rotation of the extracted product is measured.

In this present invention, modification in distillation process over prior art processes is made in order to achieve final product with specific laevo-symmetry isomeric (optical rotation) activity between −12° to −14°. The product of specific optical rotation value remains constant for a longer period of time: hence effectiveness of the products prepared from extracted terpineol will increase.

### BRIEF DESCRIPTION OF THE DRAWINGS

FIG. 1: Results of Gas Chromatography.

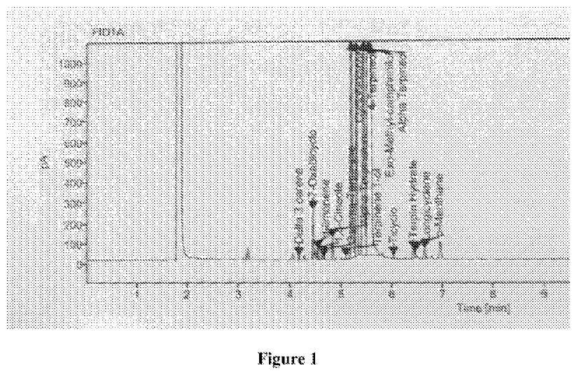

FIG. 2: Purity value of alpha Terpineol.

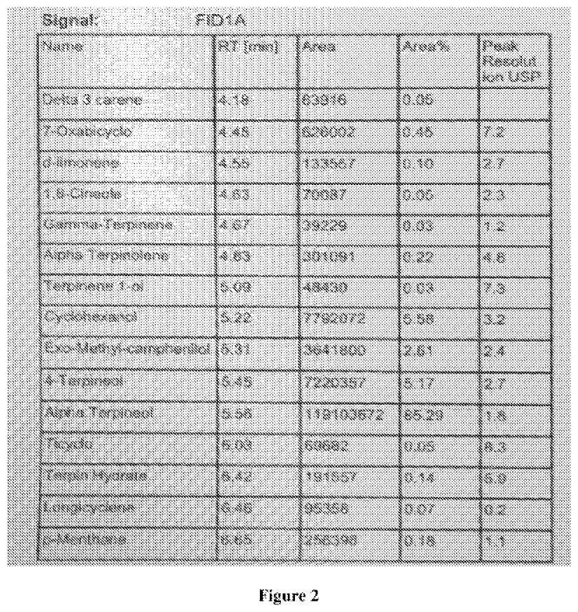

### DETAILED DESCRIPTION OF THE INVENTION

The following description is of best-contemplated mode of carrying out the invention. The description is made for the purpose of illustrating the general principles of the invention and should not be taken in a limiting sense.

The basic principle involved in the extraction of terpineol is that the conversion of turpentine specially α-pinene compound into terpineol usually takes place by hydration of the double bond functional group present within pinene structure i.e., by addition of one water molecule. This reaction starts with the transformation of one double bond into single bond of C—C to form carbocation with the help of an acid catalyst. These carbocations are further attached by water molecules forming a hydroxyl bond and become terpineol. Along with terpineol certain other by products such as borneol, cineol, fenchol etc also formed.

The present invention discloses an industrial process for manufacturing of Terpineol from gum Turpentine oil with controlled optical rotation. The process disclosed in present invention results in production of terpineol with optical rotation value ranging from −12° to −14°, which is not disclosed by any of the prior art processes disclosed for terpineol production. The disclosed process is simple and quite ecofriendly.

In the present invention, the process is modified as below:
the step of distillation is reduced to one step whereas other usual processes involve multiple steps in chain reaction with reactants.
Controlled boiling points during fine distillation, results in the production of terpineol with specific optical rotation range.

The process disclosed in present invention does not cause any pollution as all reactions are maintained in green environment along with that, lesser amount of water is involved in the reaction mechanism.

Further, the process disclosed in present invention is very efficient and cost effective as the recovery of the product (yield) is about 90%.

The stepwise process for production of terpineol is as follows:

Source and geographical location of raw material:

Raw material is obtained from the bark of pine trees from Himalayan regions of INDIA, China, Vietnam, Brazil, Indonesia, Portugal and Brazil.

Process Steps:
1. Separation of alpha pinene from gum turpentine oil through distillation.
2. Reaction of alpha pinene with Orthophosphoric acid (Acid catalyst) under a temperature ranging from 50-60 degree Celsius for about 18-20 hours, in order to produce Crude terpineol
3. Subjecting crude terpineol to distillation at 75 to 80° C. for about 50 to 60 hours to separate final product terpineol from crude Terpineol followed by measuring of boiling point and optical rotation of final product terpineol on an hourly basis.
4. Determination of Yield and purity with the help of Gas Chromatography technique. Yield is observed as >90% with respect to input raw material by w/w calculation and Gas chromatography reveals the purity of the final product that is >85%.

The product obtained with present invention was duly compared with other commercially available Terpineol of other vendors.

The results are seen as below:

TABLE 1

| | optical Rotation | optical Rotation | |
|---|---|---|---|
| Vendor Comparison | @20° C. | @25° C. | Remarks |
| A | 7.213 | 7.22 | Dextro |
| B | −69.998 | −71.303 | Levo |
| Present product | −14° | −14° | Levo |

Normally optically Active Alpha Terpineol Production by Hydration of Optically active Alpha and/or beta pinene using specified acid mixture.

The process involves the use of water, sulphuric acid, and Acetic acid etc. The raw materials are filled in 21 stirrer and heated at 100° C., reflux for 4 hours. The aqueous phase after reaction is separated and the organic phase is washed with 80 g of water and buffered with 50% sodium hydroxide. The final products are further analyzed using Gas chromatography and finally Terpineol is separated.

The process disclosed in present invention is simple and ecofriendly as it comprises of one distillation step instead of multiple steps in chain reaction with reactants. Fine distillation steps with respect to control boiling points of the compound results in obtaining optical range of −12 to −14° C.

### Novelty, Inventive Step and Industrial Application

### Novelty

The Novelty of the present invention lies in the disclosure of an industrial process used for the extraction of terpineol from Gum turpentine oil with a specific range of optical rotation value i.e. −12° to −14°. The final product Terpineol with this optical rotation value is not disclosed in any of the prior art manufacturing process, hence. This invention is unique and is still unpublished in view of patents disclosed, book publications, and also online commercial information disclosure, which makes it novel.

### Inventive Step

Inventive step by the way of technological advancement lies in modification of distillation process. Normally multiple distillation steps are used whereas the present invention utilizes distillation only at starting point to extract crude Terpineol from raw material and then after adding Orthophosphoric acid catalyst. Use of orthophospheric acid as catalyst at first stage is facilitating the reduction of distillation steps. Only one distillation process at controlled boiling point, is carried out instead of multiple steps in chain reaction with reactants.

This results in production of terpineol with controlled optical rotation value.

### Industrial Application

Industrial application is duly clear as present invention helps in production of terpineol with controlled optical rotation value i.e. −12o to −14o, which remains constant for a longer period of time and further results in increasing the effectiveness of the products produced using terpineol. Terpineol has fragrance similar to lilacs, hence is most commonly used in manufacturing of perfumes, cosmetics, aromatic scents. Also alpha terpineol possesses certain biological activities such as antioxidant, anticancer, anticonvulsant, antiulcer, antihypertensive etc.

We claim:

1. Terpineol from Gum turpentine oil WHEREIN the same has controlled Optical rotation value/Laevo-symmetry isomeric (optical) activity in the range of −12° to −14°.

2. The Terpineol from Gum turpentine oil as claimed in claim 1 WHEREIN the purity of the Terpineol >85%.

3. The method of preparing Terpineol from Gum turpentine oil as claimed in claim 1 WHEREIN the same consists of following steps:
Separating alpha pinene from gum turpentine oil through distillation;
Reacting alpha pinene with Orthophosphoric acid (Acid catalyst) under a temperature ranging from 50-60 degree Celsius for about 18-20 hours, in order to produce Crude terpineol;
Subjecting crude terpineol to distillation at 75 to 80° C. for about 50 to 60 hours to separate final product terpineol, followed by measuring of boiling point and optical rotation of final product terpineol on an hourly basis.

4. The method of preparing Terpineol from Gum turpentine oil as claimed in claim 1 WHEREIN the yield is >90% (w/w).

5. The Terpineol from Gum turpentine oil as claimed in claim 1 WHEREIN the Optical rotation value/Laevo-symmetry isomeric (optical) activity in the range of −12° to −14° remains constant for a longer period of time resulting in increasing the effectiveness of the products produced using said terpineol.
